(19) United States
(12) Patent Application Publication
(10) Pub. No.: US 2018/0199928 A1
(43) Pub. Date: Jul. 19, 2018

(54) SURGICAL ELEVATOR

(71) Applicant: Raymond Manuel Avena, Santa Clara, CA (US)

(72) Inventor: Raymond Manuel Avena, Santa Clara, CA (US)

(21) Appl. No.: 15/408,288

(22) Filed: Jan. 17, 2017

(51) Int. Cl. A61B 17/02

(52) U.S. Cl. CPC A61B 17/0218; A61B 2017/00787; A61B 2217/005

(57) ABSTRACT

A surgical elevator includes an elevator body having an elevator body tip. The surgical elevator body and the elevator body tip are adapted to provide an elevation function during a surgery. A suction tube is integrated with the elevator body. The suction tube has a suction end. A control pad is attached to the suction tube. The control pad is adapted to position the suction end of the suction tube with respect to the elevator body tip of the elevator body so that, when no suction is required, the suction end of the suction tube is retracted behind the elevator body tip of the elevator body. When suction is required, the suction end of the suction tube is positioned in front of the elevator body tip of the elevator body.

### BACKGROUND

A surgical elevator is a surgical instrument used for elevating, scraping or dissecting bones or tissue. In endoscopic microscopic ear surgery, a surgical elevator is used to elevate skin and tissue to visualize the inner ear.

### BRIEF DESCRIPTION OF THE DRAWINGS

FIG. 1 is simplified illustration of a surgical elevator in accordance with an implementation.

FIG. 2 is a simplified diagram to the surgical elevator of FIG. 1 being adjusted by a user in accordance with an implementation.

FIG. 3 is also a simplified diagram to the surgical elevator of FIG. 1 being adjusted by a user in accordance with an implementation.

FIG. 4 is simplified illustration of a surgical elevator in accordance with another implementation.

### DETAILED DESCRIPTION

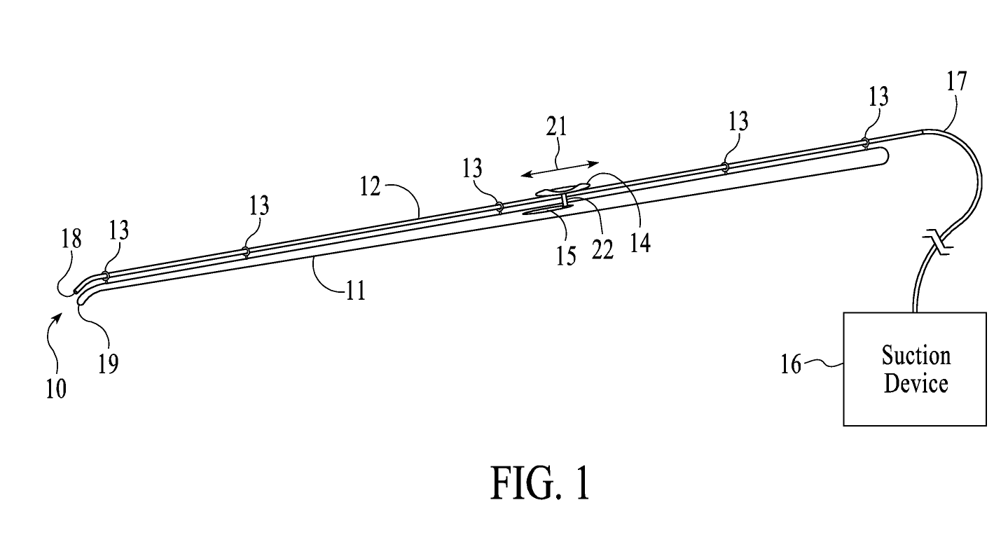

FIG. 1 shows a surgical elevator 10 that has an elevator body 11. For example, surgical elevator 10 is shaped to be used in endoscopic microscopic ear surgery. Alternatively, surgical elevator is adapted to provide an elevation function during another type of surgery. Surgical elevator 10 enables a surgeon to elevate skin and tissue necessary to visualize the inner ear. For example, when performing an ear surgery, surgeon may hold a surgical elevator in one hand while controlling a camera with another hand. When suction is required to remove blood or fluid during surgery it sometimes becomes necessary the surgeon to stop using the camera or the elevator to free up a hand to control a suction device. Cycling back and forth between using surgical elevator and using a suction device can be time-consuming, inefficient and tiring to a surgeon. Surgical elevator 10 is designed to overcome these issues.

Particularly, a suction tube 12 is integrated with elevator body 11. For example, suction tube 12 is integrated with elevator body 11 by attaching suction tube 12 to elevator body 11 in such a way as to allow a sliding movement of suction tube 12 relative to elevator body 11 as illustrated by an arrow 21. For example, a series of eye brackets 13 can be used to hold suction tube 12 to elevator body 11 while allowing movement as illustrated by arrow 21. A control pad 14 acts as a place where a surgeon can rest a finger to control sliding of suction tube 12 relative to elevator body 11. Control pad 14 is fixedly attached to suction tube 12, so that movement of control pad 14 with respect to elevator body 11 results in a corresponding movement of suction tube 12 with respect to elevator body 11.

The amount of movement allowed between suction tube 12 with respect to elevator body 11 may be restricted by a restriction device. In the implementation shown in FIG. 1, the restriction device is implemented by a band 22 attached to control pad 14 and partially located within a slot 15 of elevator body 11. The length of slot 15 and the thickness of band 22 determine boundaries of the sliding movement of suction tube 12 with respect to elevator body 11.

During surgery, suction tube 12 is connected to a suction device 16 via an extension suction tube 17. A surgeon can use control pad 14 to position a suction end 18 of suction tube 12 with respect to elevator body tip 19 of elevator body 11. For example, when no suction is required, suction end 18 of suction tube 12 is retracted behind elevator body tip 19 of elevator body 11. When suction is required, suction end 18 of suction tube 12 is positioned in front of elevator body tip 19 of elevator body 11. When designing and manufacturing surgical elevator 10, surgical elevator 10 is configured so that position of suction end 18 of suction tube 12 with respect to front of elevator body tip 19 of elevator body 11 is optimized in both a retraction position and a suction position for an anticipated surgical use of surgical elevator 10.

For example, optimization is used to assure that in a retraction position suction end 18 of suction tube 12 will not hinder use of elevator body tip 19 of surgical elevator 10 to elevate, scrape, dissect, etc. during the intended use of surgical elevator 10. Likewise, optimization is used to assure that in a suction position, suction end 18 of suction tube 12 will be in position to suction blood or other fluids without being hindered by elevator body tip 19 of surgical elevator 10.

In FIG. 1, suction end 18 of suction tube 12 is shown in a retracted position behind elevator body tip 19 of elevator body 11.

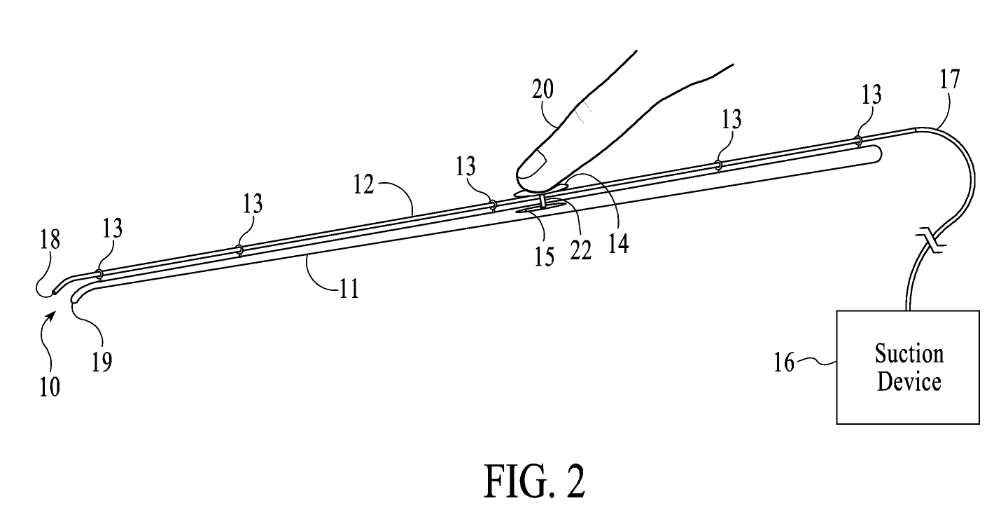

FIG. 2 shows a surgeon adjusting position of suction end 18 of suction tube 12 with respect to front of elevator body tip 19 of elevator body 11, as represented by a finger 20 of the surgeon. For example, surgical elevator 10 is configured so that while holding and using surgical elevator an index finger or a thumb finger will naturally rest upon control pad 14 facilitating positioning of suction end 18 of suction tube 12 with respect to front of elevator body tip 19 of elevator body 11. Alternatively, surgical elevator 10 may be configured so that while holding and using surgical elevator another finger will naturally rest upon control pad 14 facilitating positioning of suction end 18 of suction tube 12 with respect to front of elevator body tip 19 of elevator body 11.

In FIG. 2, suction end 18 of suction tube 12 is shown in a position midway between a retracted position, where suction end 18 of suction tube 1 is behind elevator body tip 19 of elevator body 11, and a suction position where suction end 18 of suction tube 1 is well ahead of elevator body tip 19 of elevator body 11.

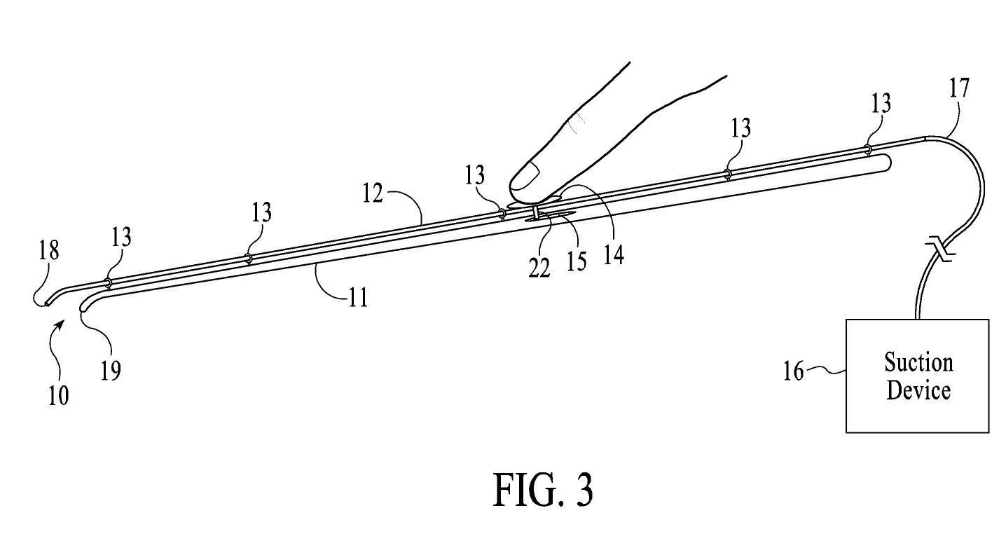

In FIG. 3, suction end 18 of suction tube 12 is shown in a suction position, where suction end 18 of suction tube 1 is well ahead of elevator body tip 19 of elevator body 11.

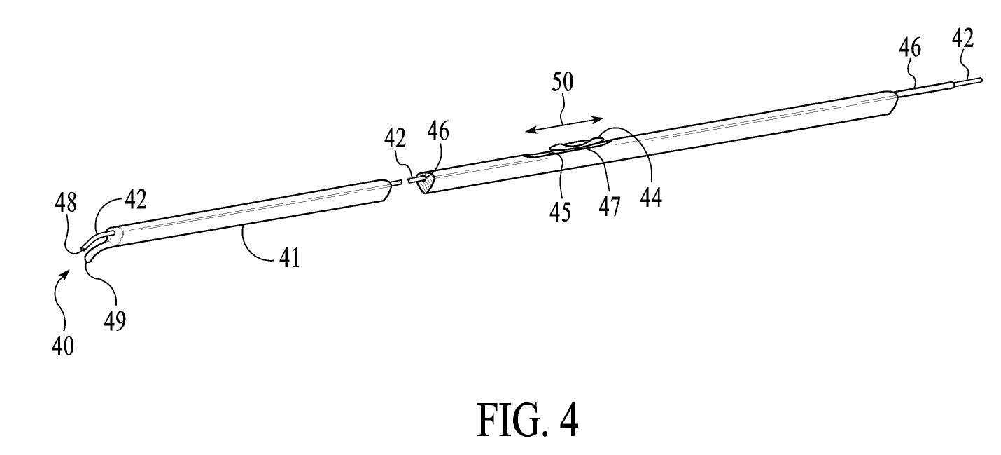

FIG. 4 illustrates the flexibility in utilizing different designs for a surgical elevator 40. In FIG. 4, a suction tube 42 integrated with an elevator body 41 by extending suction tube 42 through a hollow tube section 46 of elevator body 41. Hollow tube section 46 may be completely contained within elevator body 41, or may extend out a back of elevator body 41 as shown. Hollow tube section 46 is sized formed to allow a controlled sliding movement of suction tube 42 relative to elevator body 41, as illustrated by an arrow 50. A control pad 44 acts as a place where a surgeon can rest a finger to control sliding of suction tube 42 relative to elevator body 41. Control pad 44 is fixedly attached to suction tube 42, so that movement of control pad 44 with respect to elevator body 11 results in a corresponding movement of suction tube 42 with respect to elevator body 41.

The amount of movement allowed between suction tube 42 with respect to elevator body 41 may be restricted by a restriction device. In the implementation shown in FIG. 4, the restriction device is implemented by a band 47 attached to control pad 44 and partially located within a slot 45 of elevator body 41. The length of slot 45 and the thickness of band 47 determine boundaries of the sliding movement of suction tube 42 with respect to elevator body 41. As will be understood by persons of ordinary skill in the art, any other type of restriction device or methodology may be used.

During surgery, suction tube 12 is connected to a suction device via an extension suction tube. A surgeon can use control pad 44 to position a suction end 48 of suction tube 42 with respect to elevator body tip 49 of elevator body 41. For example, when no suction is required, suction end 48 of suction tube 42 is retracted behind elevator body tip 49 of elevator body 41. When suction is required, suction end 48 of suction tube 42 is positioned in front of elevator body tip 49 of elevator body 41. When designing and manufacturing surgical elevator 40, surgical elevator 40 is configured so that position of suction end 48 of suction tube 42 with respect to front of elevator body tip 49 of elevator body 41 is optimized in both a retraction position and a suction position for an anticipated surgical use of surgical elevator 40. The surgical use can be for ear surgery, or for another type of surgery where suction is required in addition to elevation, scraping, dissecting or some other use of a surgical elevator.

The foregoing discussion discloses and describes merely exemplary methods and embodiments. As will be understood by those familiar with the art, the disclosed subject matter may be embodied in other specific forms without departing from the spirit or characteristics thereof. Accordingly, the present disclosure is intended to be illustrative, but not limiting, of the scope of the invention, which is set forth in the following claims.

What is claimed is:

1. A surgical elevator comprising:
an elevator body having an elevator body tip, the surgical elevator body and the elevator body tip being adapted to provide an elevation function during a surgery;
a suction tube integrated with the elevator body, the suction tube having a suction end; and
a control pad attached to the suction tube, the control pad adapted to position the suction end of the suction tube with respect to the elevator body tip of the elevator body so that, when no suction is required, the suction end of the suction tube is retracted behind the elevator body tip of the elevator body and when suction is required, the suction end of the suction tube is positioned in front of the elevator body tip of the elevator body.

2. A surgical elevator as in claim 1 wherein the surgery is endoscopic microscopic ear surgery.

3. A surgical elevator as in claim 1 wherein the suction tube is integrated with the elevator body using a plurality of eye brackets.

4. A surgical elevator as in claim 1 wherein the suction tube is integrated with the elevator body by extending the suction tube through a hollow tube section of the elevator body.

5. A surgical elevator as in claim 1 additionally comprising:
a band attached to the control pad, the band extending into a slot within the elevator body, a length of the slot being chosen to restrict an amount the suction tube can be slid with respect to the elevator body.

6. A surgical system comprising:
a suction extension tube;
a suction device that produces suction within the suction extension tube; and,
a surgical elevator, comprising:
an elevator body having an elevator body tip, the surgical elevator body and the elevator body tip being adapted to provide an elevation function during a surgery,
a suction tube attached to the suction extension tube and integrated with the elevator body, the suction tube having a suction end, and
a control pad attached to the suction tube, the control pad adapted to position the suction end of the suction tube with respect to the elevator body tip of the elevator body so that, when no suction is required, the suction end of the suction tube is retracted behind the elevator body tip of the elevator body and when suction is required, the suction end of the suction tube is positioned in front of the elevator body tip of the elevator body.

7. A surgical system as in claim 6 wherein the surgery is endoscopic microscopic ear surgery.

8. A surgical system as in claim 6 wherein the suction tube is integrated with the elevator body using a plurality of eye brackets.

9. A surgical system as in claim 6 wherein the suction tube is integrated with the elevator body by extending the suction tube through a hollow tube section of the elevator body 41.

10. A surgical system as in claim 6, wherein the surgical elevator additionally comprises:
a band attached to the control pad, the band extending into a slot within the elevator body, a length of the slot being chosen to restrict an amount the suction tube can be slid with respect to the elevator body.

11. A method for combining a suction function with an elevator function is a surgical tool, the method comprising:
integrating a suction tube having a suction end with an elevator body having an elevator body tip, where the surgical elevator body and the elevator body tip are adapted to provide an elevation function during a surgery; and,
attaching a control pad to the suction tube so that the control pad is adapted to position the suction end of the suction tube with respect to the elevator body tip of the elevator body so that, when during a surgery no suction is required, the suction end of the suction tube is retracted behind the elevator body tip of the elevator body and when suction is required, the suction end of the suction tube is positioned in front of the elevator body tip of the elevator body.

12. A method as in claim 11, wherein the surgery is endoscopic microscopic ear surgery.

13. A method as in claim 11, wherein the suction tube is integrated with the elevator body using a plurality of eye brackets.

15. A method as in claim 11, wherein the suction tube is integrated with the elevator body by extending the suction tube through a hollow tube section of the elevator body 41.

15. A method as in claim 11, additionally comprising:
attaching to the control pad so that the band extends into a slot within the elevator body, where a length of the slot restricts an amount the suction tube can be slid with respect to the elevator body.
